(19) United States
(12) Patent Application Publication
(10) Pub. No.: US 2025/0073182 A1
(43) Pub. Date: Mar. 6, 2025

(54) LOW DOSAGE GOSSYPOL CONTRACEPTIVE FOR RODENTS

(71) Applicant: SENESTECH INC., Phoenix, AZ (US)

(72) Inventors: Courtney RAY, Gilbert, AZ (US); Amber JANUSZ, Glendale, WI (US); Bill ADAMS, Phoenix, AZ (US); Connor STEWART, Phoenix, AZ (US); Alexander MATUSZEK, Glendale, AZ (US); Dean HEYL, Sun City, AZ (US); Sandeep KAUR, Surprise, AZ (US)

(73) Assignee: SENESTECH INC., Phoenix, AZ (US)

(21) Appl. No.: 18/823,243

(22) Filed: Sep. 3, 2024

Related U.S. Application Data
(60) Provisional application No. 63/580,233, filed on Sep. 1, 2023.
(60) Provisional application No. 63/590,848, filed on Oct. 17, 2023.

(51) Int. Cl. A61K 31/11; A61K 9/00; A61K 47/12; A61K 47/46; A61P 15/16; A61P 15/18

(52) U.S. Cl. CPC A61K 31/11; A61K 9/0056; A61K 36/577; A61K 47/12; A61K 47/46; A61P 15/16; A61P 15/18

(57) ABSTRACT

An orally ingestible contraceptive low dose gossypol composition for rodents such as mice, rats and members of the order Rodentia is provided. The contraceptive composition comprises 0.0005 to 0.0045 wt. % of gossypol and a carrier. The carrier includes a bulk feed component. The bait or soft bait may be encased in a casing such as a collagen casing. Also provided is a method for contraception of rodents comprising administering the low dose-gossypol composition by making the contraceptive available for feeding the target animal.

### REFERENCE TO A RELATED APPLICATION

Provisional U.S. Patent Application No. 63/580,233, filed Sep. 1, 2023 and Provisional U.S. Patent Application No. 63/590,848, filed Oct. 17, 2023 are incorporated by reference for all purposes.

### BACKGROUND

### Technical Field

The present invention pertains to the domain of a wildlife fertility control product, specifically focusing on the mitigation of fertility in rodent pests through the utilization of the chemical agent, gossypol, known for reducing sperm count and motility. This invention encompasses formulations, systems, and methodologies for control of rodents, including Rattus norvegicus, Rattus rattus, Sigmodon hispidus, and Oryzomys palustris, Mus musculus, voles, and gophers by reducing fertility.

### Related Art

Rapid proliferation of rodent populations particularly can trigger a range of ecological and environmental challenges. These include, but are not limited to, disturbances to forest, grassland, and wetland ecosystems, and implications for lands designated for agriculture or grazing. In urban settings, rodents frequently undermine the integrity of structures and habitats, resulting in habitat degradation and eventual loss for various species. Furthermore, damages extend to farms, ranches, beaches, and even recreational sites like campgrounds, parks, and golf courses. Economically, these rodent pests pose a significant threat to various crops including grains, vegetables, fruits, and nuts, thereby impacting the revenues of farmers. The unchecked growth of feral rodent populations has led to escalating damage across farms, ranches, beaches, golf courses, and beyond. With the capability to produce multiple litters annually, these rodents can quickly overrun properties such as farms or ranches. However, removing food sources and sanitizing to fend off adaptable and intelligent rodents from farms or residential areas are both complex and financially burdensome.

Moreover, rodents are potential carriers of diseases and can serve as vectors or reservoir of viruses and other microbes. However, overly aggressive reduction in numbers of rodents may be undesirable. Elimination of too many rodents might trigger unforeseen ecological consequences. For instance, rodents contribute to biodiversity through seed dispersal, scavenging, organic material recycling, and insect and other pest population control. Moreover rodents represent part of the diet for predators like owls and snakes. Thus total elimination or overly zealous rodent population reduction is not desirable.

Rodenticides have been used for many years to attempt to control populations of rodent pests. However, rodent populations have been difficult to control resulting in continuing rodent damage, spoilage, and risk of disease transmission. Rodents have short gestation periods in the order of weeks and thus unless an entire population is eliminated, the rodent population is quickly restored once the rodenticide is depleted. Moreover rodenticides are poisonous to non-targeted animals and humans and lack specificity for rodent pests such as rats and mice. Non-specific poisoning of non-target animals raises serious environmental concerns. While poisoning reduces a rodent population, there is less competition for food in the resulting reduced population, letting survivors reproduce more rapidly.

The inventors considered compositions that reduce the reproductive capacity of rodent pests to be desirable for a variety of reasons. Such compositions may be used to induce infertility in these pests thus reducing and controlling rodent populations.

Previous work has proposed the use of gossypol, a contraceptive compound found in cotton seeds, as a contraceptive agent for certain animals, see US2023/0016955 and US20220233467. Gossypol is known for its antifertility effects which can vary depending on purity, dose, duration, route of administration, and the species of test animal (Nowzari et al. 2021; Romualdo et al. 2022). However, like rodenticides, gossypol's toxicity isn't exclusive to rodents and can affect a broader range of animals. Moreover, gossypol is a bitter compound and high levels in a rodent bait can deter bait consumption.

In female rats, gossypol has an endocrine effect on the ovary and cytotoxic effect on early embryo development, resulting in longer estrous cycle due to an increase in the time spent in diestrus and disrupted pregnancy (Gadelha et al. 2014; Lin, et al., 1985). Gossypol affects ovarian follicular maturation and significantly decreased the number of viable follicles and increased the number of atretic follicles for all stages of development in female mice when provided in vitro (Luz et al. 2018; Ding et al. 2021). Research shows gossypol inhibits embryo implantation in the early stages of pregnancy and may have dose dependent embryocidal effects when provided post-implantation (Sein, 1986; Li et al. 1989; Lin et al. 1989). Additionally, toxicity in females is observed at lower doses than males but males are more sensitive to the antifertility effects of gossypol at low concentrations.

The effects of gossypol on male rodent reproduction are inhibited spermatogenesis, reduced sperm counts, and sperm immobility (Randel et al. 1992; El-Sharaky et al. 2010; Chris-ozoko et al. 2018; Fornes et al. 1993; Ali et al. 2010). When lesser bandicoot rats, Bandicota bengalensis, ingested 0.1% cottonseed oil mixed into their food source at a ratio of 5% and 10%, female reproduction was affected and males showed significantly reduced sperm viability and motility (Singla and Garg, 2013). Male mice provided 20 or 40 mg/kg body weight (BW) of gossypol had a significant decrease in seminal vesicle weight with non-uniform regressive changes found in the seminiferous tubules of the testis following 3 weeks of gossypol treatment (Singh and Rath, 1990). Hunt and Mittwoch (1984) showed reduced sperm counts for two different mouse strains after 10 and 20 mg/kg BW of gossypol was administered orally for 4 weeks.

Administration of gossypol at low doses caused defects in over 50% of the spermatozoa collected from males after only 4 weeks and no offspring were produced (Nadakavukaren et al. 1979). Similarly, there were significantly lower pregnancy rates in as early as 6 weeks when male rats were given ≥10 mg/kg/d of gossypol and bred to fertile females (Randel et al. 1992). Testicular effects of cottonseed oil on male mice is known to cause spermatogenesis arrest and sperm abnormalities at relatively low doses (Farrag et al. 2007). Chang et al. (1982) demonstrated that responses to gossypol are both dose and time dependent. Epididymal and accessory sex glands weights were reduced when rats were fed gossypol daily at the rates of 30 mg/kg for 6 weeks or 7.5 and 15 mg/kg for 12 weeks. Fertility was reduced and toxicity absent in rats following consumption of gossypol in cottonseed meal at 15 mg/kg per day for 4 weeks. Additionally, Coulson et al. 1980 showed that low gossypol dosing provided to male mice for extended periods of time can produce similar reproductive effects seen with high doses. Based on these data, the inventors sought to formulate a soft, palatable bait containing a low palatable concentration of gossypol that would reduce the fertility of male and female rodents without side-effects when consumed over a period of time while minimizing negative effects on the environment and on other non-target species from lethal rodenticides.

Due to these challenges the inventors recognized a need for a stable, castable and attractive bait having a lower gossypol dose than current baits to control populations of wild rodents while minimizing environmental impacts that lethal means of controlling these populations pose.

### SUMMARY OF THE INVENTION

A primary facet of this disclosure revolves around a composition containing 0.0005-0.0045 wt. % unbound gossypol or free gossypol. This composition also includes a carrier consisting of bulk feed elements. An exemplary formulation comprises oat flour, cottonseed meal, cottonseed oil, shortening, gossypol (concentration between 0.0005 and 0.0045 wt. %), sodium saccharin (or other artificial sweetener), and citric acid (or other preservative). Gossypol content of this composition is provided by the gossypol found in cottonseed. The composition is typically designed to be palatable for targeted rodents.

A contraceptive method is introduced, focusing on the administration of a composition containing a low dose of gossypol to a targeted male or female rodent.

Another aspect of the invention is a soft bait for rats and other small rodents that is encased in a casing such as a collagen casing.

### BRIEF DESCRIPTION OF THE DRAWINGS

FIGS. 1A, 1B, and 1C depict a low dose gossypol bait according to the invention. SenesTech's gossypol bait is a soft dough consisting of flour and oil or shortening, which can be sold in various sizes and packaging materials and deployed in locking rodent bait stations such as that pictured or broadcast.

FIG. 2. Rodent consumption of bait having free gossypol content ≤0.025 wt. %; see Example 1.

FIG. 3A-3C. Reproductive effects following consumption of bait having free gossypol concentration of <0.0025 wt. %; see Example 2.

FIGS. 4A-4C show embodiments of the encased edible soft baits of the invention; see Example 3.

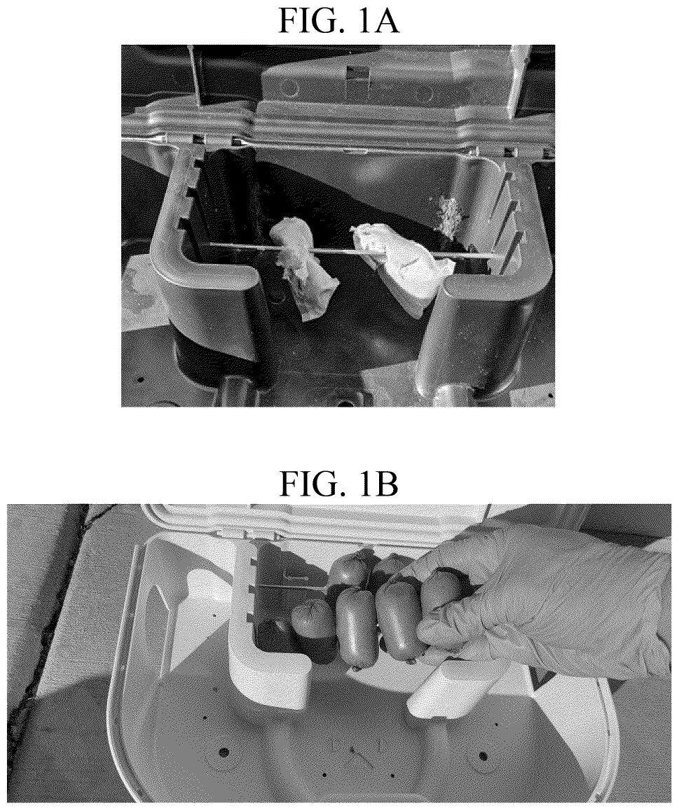

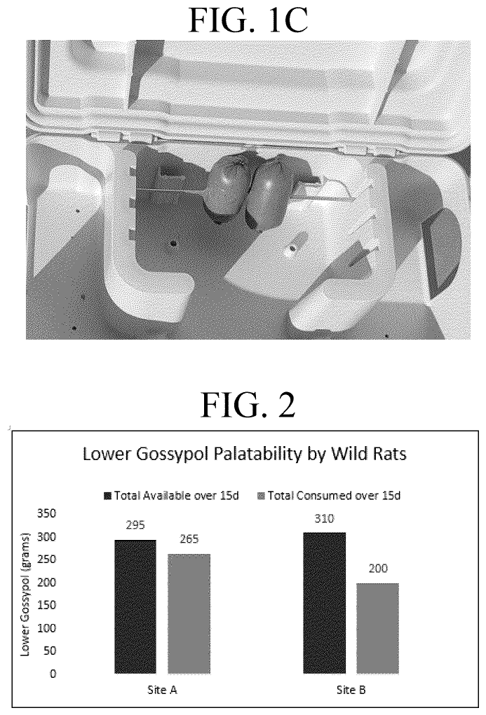

### DETAILED DESCRIPTION OF THE INVENTION

### Terminology

Rodents: This term includes, but is not limited to, mice including Mus musculus, rats including Norway rat and roof rat, squirrels, gophers, beavers, pacas, chipmunks, and other members of the order Rodentia. It includes but is not limited to various species of rats such as Rattus norvegicus, Rattus rattus, Sigmodon hispidus, and Oryzomys palustris, and various types of mice, such as is Mus musculus, Peromyscus maniculatus, Peromyscus leucopus, Reithrodontomys megalotis, or Peromyscus gossypinus.

Gossypol: Encompasses free gossypol, bound or non-free gossypol such as gossypol bound to protein, racemic gossypol, (+) gossypol, (−) gossypol, aldehyde, ketone, and lactol gossypol tautomers, and its derivatives like gossypolone, apogossypol, or apogossypolone. It also covers biologically active gossypol as well as inactive gossypol, and its derivatives. A representative gossypol structure is provided:

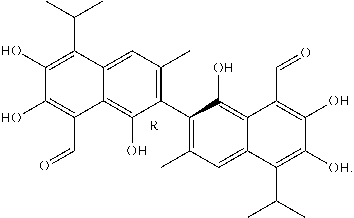

Carrier Composition: Preferred compositions comprise oat flour, cottonseed meal, and cottonseed oil, the latter two can contribute gossypol. Other natural ingredients may form the carrier or parts of it such as grains, seeds, pasta, flours, doughs, or pastes. A carrier may comprise fish oils, molasses, peanut butter and other flavorings. In some embodiments, sweet or sugary ingredients, dairy products, meat or fish ingredients are excluded so as not to attract non-target animals or insects. The carrier may contain a preservative or stabilizer such as ethyl cellulose, gelatin, collagen, gum Arabic, carrageenan, borates, sorbic acid, propionic acid, benzoic acid or salts thereof, or waxes or other agents that prevent or reduce mold, fungal, or other microbial contamination.

Bait softness. This invention comprises a combination of rodent fertility control with soft, palatable bait that would be unique in the market. A typical soft bait is pliable and soft under the conditions of its uses, for example, a soft bait can be formulated to be soft and pliable under different environmental temperatures or in different geographical locations. A soft bait may resemble a dough or paste and have a texture that allows the target animal to bite or consume it. One measure of softness is the ability of a rodent to consume the bait and consume an amount sufficient to induce a contraceptive effect. Another measure of hardness or softness is provided by the Shore Hardness Scales, for example, by Shore 00 which characterizes extra soft (0, 10, 20, 30, 40, 50), soft and medium soft foods (>50, 55, 60, 65, 70, 75, 80, 90). The compositions disclosed herein preferably have a softness corresponding to extra soft, soft, and medium soft. In some embodiments, the compositions may be formulated as harder products outside this range, e.g., having a Shore 00 value of >90.

In some embodiments, the bait is formulated as a pellet or kibble, such as a pellet having an average diameter ranging from 1, 2, 3, 4, 5, 6, 7, 8, 9, or 10 mm or a size suitable for carriage and/or consumption by mice or rats. The pellets or kibbles may be produced by extruding and or compressing a bait into a uniform size and optionally drying the compressed or extruded bait or applying a protective coating to prevent damage by moisture or contamination by insects, molds, or bacteria. In some embodiments, the pellets or kibbles will be filled into containers, such as rigid plastic containers or plastic bags.

Soft baits typically are formulated to contain similar concentrations of an active agent like a contraceptive and to have a uniform texture.

Soft baits often contain agents such as scents, flavors, pheromones, or additives that entice a target animal to consume it.

Soft baits may be packed inside of casings or have a sausage like shape. They may be in a discrete form such as a cube or pellet that in some embodiments is packed or filled into a casing. A pellet or cube or rectangular prism typically has a size that can easily be packed into a casing, for example, an average diameter or length width or height (for cubes or prisms) about ≤1, 1, 2, 5, 10, 20, 50 or 100% the diameter of the casing (or any intermediate value within this range).

A dosage of active ingredient, such as a contraceptive ingredient like gossypol, in a soft bait may be selected to produce a multistage soft bait. Such a multistage bait typically has a lower potency compared to a pulse bait that administers an effective dose at one time. A multistage bait usually reduces the risk of administering the active agent such as gossypol to non-target animals as it must be consumed over a longer period of time for efficacy. Due to a lower concentration of the active ingredient target animals are less likely to avoid baits containing bitter or distasteful amounts of an active ingredient (bait shyness).

Bait Casings are protective enclosures that contain or house a soft bait. It provides a barrier to protect the bait from the environment and from microorganisms, insects and other pests. It also may be used to control the rate of feeding of a rodent or other target animal for example by preventing the bait from being too easily accessed or removed by the target animal. It protects the bait from moisture, contamination, hardening or degradation. Advantageously the casing is edible. It may be designed or configured to make the bait more attractive to the target animal, for example, by incorporation of scents, flavorings, or visual cues. A casing may be selected or formulated to inhibit non-targeted animals from consuming the bait. In some embodiments, the bait casing is selected to permit pasteurization of sterilization of the soft bait inside or to facilitate irradiation to kill microorganism.

To enhance stability of a soft bait, the casing provides a physical barrier from outside conditions including exposure to sunlight, rain, moisture or high humidity, from drying and hardening, or from contamination by dust or soil or by microorganisms or insects.

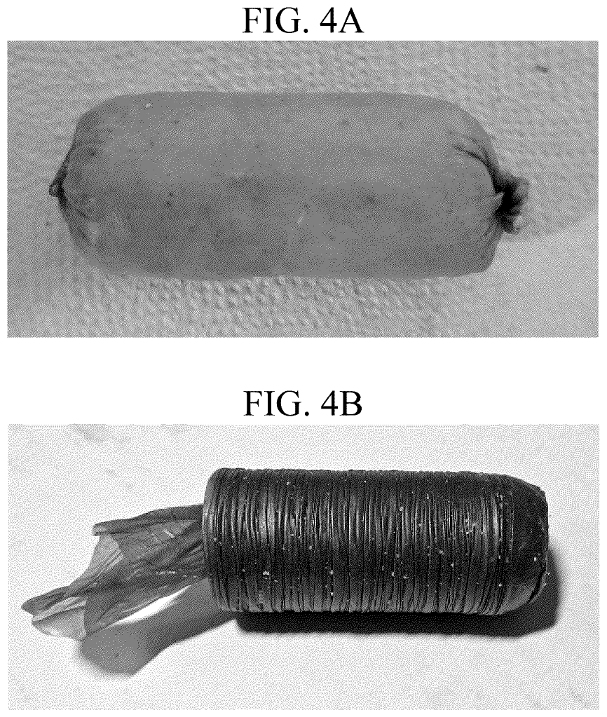

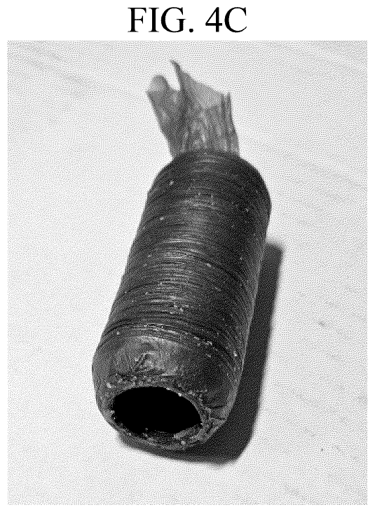

Preferably, the casing is configured to permit odors from a bait. This helps lure an animal to a bait and enhances bait consumption. For example, twists on the end of a casing enclosing a bait can be configured to permit the target animal to smell the bait inside while protecting the bait from the external environment. In some embodiments, the filled casing comprises one or more links, such as those described by FIGS. 4A-4C. A link may be closed, for example, by twisting at one or both ends (FIG. 4A) or may be left at least partially open an another end as shown by FIGS. 4B and 4C. A link may have a concentric opening at one or both ends. The distal end diameter of the opening based on the diameter of the link may range from 1, 2, 5, 10, 20, 30, 40, 50, 60, 70, 80, 90, 95, or 100% of the diameter or average diameter of the link. The opening may be tapered from the body of the link to the end opening.

In some embodiments the a cross-section of body of a filled casing may be triangular, square, rectangular, hexagonal, or other regular or irregular polygon. Nonconcentric or concentric openings at the ends of a link may comprise 1, 2, 5, 10, 20, 30, 40, 50, 60, 70, 80, 90, 95, or 100% of the area of a section of the body of the link.

Casings may be pliable and soft enough for the target animals to bite into thus revealing the soft bait inside for consumption. Suitable types casing materials include collagen (primary), natural, cellulose, polysaccharide, synthetic and cotton.

In one embodiment an edible or not edible collagen casing is used to encase the soft bait. The edible casing can be from collagen from the inside layer of beef hides. It usually does not require wetting and can be extruded to an exact size to encase an appropriate portion of the soft bait. It can be used to encase bait that is later heat treated or cooked. The edible and digestible collagen coating is typically thinner than the non-edible collagen casing. A collagen film for use in a collagen casing may be made and evaluated as described by Liu, F. et al., Changes in Structures and Properties of Collagen Fibers during Collagen Casing Film Manufacture. FOODS 2023, 12(9), 1847 which is incorporated by reference. Collagen casings are also commercially available, for example, those available from Walton's at <hypertext transfer protocol secure://waltons.com/categories/collagen-casingsor those available at <Amazon.com> both of which are incorporated by references and last accessed on Oct. 16, 2023.

Natural casings include those made from the intestines of bovines, sheep, and pigs. Typically, these are edible casings. Natural casings usually require more preparation such as cleaning and soaking prior to use and require refrigeration.

Polysaccharide casing include cellulose casings made from plant-based cellulose, alginate casings, and chitosan casings. Typically, these are edible casings.

Synthetic casings include those made from cellulose and collagen, mentioned above, and plastics.

Cotton casings can impart breathability allowing for release of attractive bait odors. They also typically have a high degree of uniformity to the encased bait, are heat stable, and provide hygienic protection to a bait.

Casing diameters and lengths are based on the size and type of target animal and to provide access to a sufficient amount of bait to the target animal.

Casing diameters may range from <15, 15, 16, 17, 18, 19, 20, 21, 22, 23, 24, 25, 26, 27, 28, 29, 30, 31, 32, 33, 34, 35, 36, 37, 38, 39, 40, 41, 43, 43, 44, 45, or >45 mm. In one preferred embodiment the casing diameter is about 26 mm.

The thickness of the surface or membrane of a casing may range from 0.1, 0.2, 0.5, 1, 1.5, 2.0, 2.5 to 3.0 mm in thickness or average thickness.

Casing length can range up to 6 inches or more, for example, <0.5, 0.5, 1, 2, 3, 4, 5, or 6 inches. In one preferred embodiment, the casing length is about 2 inches.

Casing colors may be selected based on visual appearance to the target animal or based on marketing and labelling factors, for example, to distinguish encased baits from each other based dosage or identity of a target animal or for commercial identification. Colors including mahogany, red, or clear.

Compositional Embodiments. The following non-limiting embodiments describe certain aspects of the invention.

A composition comprising, consisting essentially of, or consisting of 0.0005, 0.0006, 0.0007, 0.0008, 0.0009, 0.001, 0.002, 0.003, 0.004 to 0.0045 wt. % free gossypol; and a carrier comprising a bulk feed component. A composition preferably has a sufficient concentration of free gossypol to produce a contraceptive effect in mice or rats when consumed in one or multiple feedings. Preferably, the free gossypol concentration provide a bait that when consumed by non-target animals induces no or minimal contraceptive effect.

In typical embodiments, this composition is formulated for oral ingestion of a contraceptive inducing amount by a targeted rodent.

In a preferred embodiment, this composition comprises oat flour, cottonseed meal, shortening, cottonseed oil, sodium saccharin, and citric acid. The inventors recognized that oat flour has several advantages recommending it for inclusion in a rodent bait and it is easily molded or cast into a shape suitable for consumption by a rodent.

A preferred embodiment of the low dose gossypol bait of the invention comprises cottonseed meal and cottonseed oil as essential ingredients, but permits substitution of oat flour with wheat flour, peanut flour, coconut flour, almond flour, buckwheat flour, chickpea flour, corn flour, rice flour, tapioca flour, or any grain or rice flour or mixtures thereof. In this preferred embodiment, the shortening may be selected from fully hydrogenated oils from olive, peanut, canola, sunflower, palm kernel oil, or coconut oil and the sweetener may be selected from aspartame, neotame, and/or Acesulfame K. Also in this preferred embodiment, citric acid may be substituted with BHT, Sodium Benzoate, sorbic acid, BHA, TBHQ, phenoxyethanol, wheat germ oil, (Vitamin E), Rose Hips (Vitamin C), salt or mixtures thereof.

In some embodiments the composition has a free gossypol concentration ranging from 0.0005 to 0.0045 wt. % and in other embodiments has a free gossypol concentration ranging from 0.0005 to 0.0025 wt. %.

In some embodiments the gossypol content of the composition is derived from inclusion of cottonseed or cottonseed components such as cottonseed meal or cottonseed oil. Other cottonseed-derived materials include cottonseed per se, crushed cottonseed, hulled cottonseed, and defibered cottonseed In other embodiments, the gossypol content is derived from inclusion of another natural source of gossypol such as from okra.

In preferred embodiments, the soft bait contains a concentration of gossypol that when ingested by a target animal is non-lethal, thus reduce adverse environmental effects that accompany substantially total extermination of target animals. In some embodiments, the cottonseed comprises 1, 2, 3, 4, 5, 6, 7, 8, 9, 10, 11, 12, 13, 14, 15, 16, 17, 18, 19, 20, 30, 40, 50 or >50 wt. % of the composition.

In some embodiments, the cottonseed meal comprises 1, 2, 3, 4, 5, 6, 7, 8, 9, 10, 11, 12, 13, 14, 15, 16, 17, 18, 19, 20 or >20 wt. % of the composition. In some embodiments the cottonseed oil comprises 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5 or >5 wt. % cottonseed oil.

In some embodiments the composition comprises 10, 20, 30, 40, 50, 60, 70, 80 or >80 wt. % oat flour, preferably from 60-80% oat flour

In other embodiments, the composition comprises 0.1, 0.2, 0.3, 0.4, 0.5, 1, 2, 3, 4, or 5 wt. % sodium saccharin or another artificial sweetener, such as aspartame, sucralose, acesulfame, or steviol. Alternatively or in addition to the artificial sweetener, sugar, molasses, high fructose corn syrup, or other nutritive sweeteners may be incorporated.

In some embodiments <0.01, 0.01, 0.02, 0.05, 0.1, 0.2, 0.25, 0.5 or 1 wt. % citric acid are incorporated into the composition disclosed herein. Alternatively or in addition to the citric acid component, malic acid, tartaric acid, sorbic acid, acetic acid, propionic acid, benzoic acid or potassium sorbate may be added in similar amounts to adjust pH, enhance flavor, or as preservatives.

In some embodiments, the cottonseed or cottonseed meal when used contains 0.01, 0.02, 0.05, 1, 2, 3, 4, 5, 6 to 7 wt. % free gossypol.

In some embodiments, the cottonseed oil contains up to 0.1 to 0.2 wt. % free gossypol.

Another aspect of the invention is a method for contraception of a rodent comprising administering the gossypol composition to the rodent in an amount sufficient to reduce sperm count, sperm motility, or lower sperm morphology score in a male target rodent and to disrupt estrous cycle, reduce ovarian follicles, or impair early embryonic development and pregnancy in a female target rodent consuming the composition. In one embodiment, after consuming the composition the method provides a targeted male rodent that has a reduced sperm motility score, reduced sperm count, or a reduced sperm morphology score and a targeted female rodent that has disrupted estrous cycle, reduced ovarian follicles, or impaired early embryonic development and pregnancy compared to a control rodent not consuming the composition. In another embodiment, a cumulative amount of free gossypol or gossypol derivative is administered orally over a period of 1, 2, 3, 4, 5, 6, 7, 8, 9, 10, 11, 12, 13, 14 or more days that reduces male or female rodent fertility or the related phenomena described above.

As used herein, the terms “administering” or “administer” refer to feeding as well as placement of the bait composition disclosed herein in a manner that permits rodents to ingest it.

### Casing and Soft Bait Embodiments

Additional embodiments of the invention include but are not limited to those involving encasing a soft bait.

A further aspect of the invention comprises, consists essentially of, or consists of a containing soft bait for a small animal contained within, covered by, stuffed into, injected into, or filled into a casing. Advantageously the bait may be one suitable for population control of small animals, such as mice, rats, gophers, ground squirrels and other rodents. Any of the baits disclosed herein may be incorporated or packed into a casing. Baits include those containing contraceptives such as gossypol as well as other non-lethal contraceptives or active agents.

One embodiment of this aspect of the invention is directed to an encased orally ingestible soft bait composition for a rodent or other animal pest that comprises an orally ingestible contraceptive, a pliable bulk feed component, and a casing that encases the soft bait composition.

In some embodiments, the encased soft bait composition comprises an orally ingestible contraceptive such as gossypol. In a further embodiment, the soft bait comprises a concentration of gossypol ranging from 0.0005-0.0045 wt. % free or unbound gossypol.

In many embodiments, the casing of the encased soft bait protects it from heating, drying, contamination with dirt or soil or by insects or other conditions that would reduce the potency, allure, or edibility of the soft bait compared to an identical soft bait that is not encased.

The casings used to encase the soft baits disclosed herein may comprise a variety of different materials. In a lead embodiment, the casing comprises collagen which may be a light, edible form of collagen or a heaver less edible form.

In other embodiments the casing comprises cellulose, a polysaccharide, cotton or other woven or nonwoven fibrous casing.

The casing may also be derived from a nature course such as a natural casing derived from intestinal tissues.

In other embodiments the casing may be a synthetic or artificial material such as a plastic, synthetic fabric, or resin.

In many embodiments, the casing is edible and/or digestible. In some embodiments, the case itself, but not the soft bait it contains, is not edible or digestible.

A soft bait may be pliable at room temperature, at an outdoor temperature, or at another environmental temperature at which the soft bate is distributed. For example, the soft bait may be pliable at a temperature of 0, 10, 20, 30, 40, 50 or >50° C.

In some embodiments, the soft bait or the casing may be configured or formulated to release alluring odors from the soft bait or comprises a scent or attractant for a rodent or other target animal.

In some embodiments, the encased orally ingestible soft bait disclosed herein ranges in length from 0.5, 1, 2, 3, 4, 5, 6, 7, 8, 9 to 10 inches and in diameter or average diameter from 15, 20, 25, 30, 35, 40 to 45 mm or more.

The encased orally ingestible soft bait may comprise a casing skin or membrane that ranges from 0.1, 0.2, 0.25, 0.5, 1, 1.5, 2.0, 2.5, to 3.0 mm in thickness or average thickness.

Another aspect of the invention is directed to a method for reducing a population of rodents or other animal pests comprising placing an orally ingestible soft disclosed herein in a location frequented or accessible to the rodent or other animal pest, wherein, optionally, the soft bait emits an odor that lures or encourages its consumption of the soft bait and administering said soft bait to the rodent or other animal pest upon consumption of the soft bait.

Another aspect of the invention is directed to a method for making an orally ingestible soft bait composition for a rodent or other animal pest that comprises an orally ingestible contraceptive, a pliable bulk feed component, and a casing that encases the soft bait composition comprising: preparing a soft bait by admixing the orally ingestible contraceptive with a pliable bulk feed ingredient, filling the casing with the soft bait, and tying, fusing, gluing, or otherwise securing the ends of the casing so as to secure the soft bait inside the casing.

In some embodiments, the soft bait is encased in the casing by a single stage vacuum stuffer and in other embodiments, the soft bait is encased in the casing by a dual or multiple stage vacuum stuffer.

In other embodiments, the soft bait is encased in the casing by a horn stuffer, horizontal stuffer, vertical stuffer or hydraulic stuffer.

In a specific embodiment, the method of making the encases soft bait comprises selecting and providing a casing, providing a vacuum stuffer comprising a filling chamber, a vacuum system, and a stuffer nozzle, positioning the casing on the stuffer nozzle, introducing the soft bait into the filling chamber, activating the vacuum system to apply reduced pressure in the filling chamber, expelling air from the casing by applying the reduce pressure to the filling chamber, thereby expanding and conforming the casing to the stuffer nozzle, introducing the soft bait into the casing by applying positive pressure to the filling chamber, thereby forcing the soft bait into the casing, optionally filling a segment of the casing filed with the first soft bait with a second or subsequent soft bait, sealing the casing when filled with the soft bait(s), removing the casing from the stuffer nozzle. In a preferred embodiments, the casing used in this method comprises edible collagen.

Specific non-limiting embodiments include the following:

1. An orally ingestible contraceptive composition for a rodent, comprising:
0.0005-0.0045 wt. % free gossypol; and
a carrier containing a bulk feed component.

2. The composition of embodiment 1, formulated for oral ingestion in a contraceptive inducing amount by the targeted rodent.

3. The composition of embodiment 1, formulated for oral ingestion in a contraceptive inducing amount by Rattus norvegicus, Rattus rattus, Sigmodon hispidus, or Oryzomys palustris.

4. The composition of any one of embodiments 1-3, wherein the carrier comprises oat flour, cottonseed meal, cottonseed oil, shortening, sodium saccharin, and citric acid.

5. The composition of any one of embodiments 1-4, wherein the free gossypol concentration ranges from 0.0005 to 0.0045 wt. %

6. The composition of any one of embodiments 1-4, wherein the free gossypol concentration ranges from 0.0005 to 0.001 wt. %

7. The composition of any one of embodiments 1-6, wherein free gossypol is derived from cottonseed.

8. The composition of any one of embodiments 1-7, comprising:
0.0005 to 0.0045 wt. % free gossypol,
60-80 wt. % oat flour,
1 to 10 wt. % cottonseed meal,
0.05 to 1 wt. % cottonseed oil,
20 to 30 wt. % shortening,
0.1 to 5.0 wt. % sodium saccharin or other non-nutritive sweetener, and
0.05 to 0.25 wt. % citric acid or other preservative.

9. The composition of any one of embodiments 1-8, comprising:
0.0005 to 0.0045 wt. % free gossypol,
60-80 wt. % oat flour,
1 to 10 wt. % cottonseed meal,
0.05 to 1 wt. % cottonseed oil,
20 to 30 wt. % shortening,
0.1 to 5.0 wt. % sodium saccharin, and
0.05 to 0.25 wt. % citric acid.

10. The composition of embodiment 8 or 9, wherein the gossypol content is derived from cottonseed.

11. The composition of embodiment 8 or 9, wherein the cottonseed meal contains 0.01 to 7 wt. % free gossypol.

12. The composition of embodiment 8 or 9, wherein the cottonseed oil contains 0.1 to 0.2% wt. % free gossypol.

13. The composition of embodiment 8 or 9, wherein the shortening comprises lard, tallow, poultry fat, or vegetable shortening.

14. A contraceptive method for a rodent, comprising administering the composition of any one of embodiments 1-13 to the rodent.

15. The method of embodiment 14, wherein said administering is placing the composition at a location accessible to its consumption by the targeted rodent.

16. The method of embodiment 14 or 15, wherein an amount of the composition is administered to a male rodent that reduces sperm count, sperm motility, or sperm morphology score in the male rodent consuming or repeatedly consuming the composition.

17. The method of embodiment 14, 15 or 16, wherein the consuming rodent displays a reduced sperm motility score, reduced sperm count, or reduced sperm morphology score compared to a control rodent not consuming the composition.

18. The method of any one of embodiments 14-17, wherein the rodent is a rat.

19. The method of any one of embodiments 14-18, wherein the consuming rodent is Rattus norvegicus, Rattus rattus, Sigmodon hispidus, and Oryzomys palustris.

20. The method of any one of embodiments 14-17, wherein the rodent is a mouse.

21. The method of any one of embodiments 14-17 or 20, wherein the rodent is Mus musculus, Peromyscus maniculatus, Peromyscus leucopus, Reithrodontomys megalotis, or Peromyscus gossypinus.

22. An orally ingestible soft bait composition for a rodent or other animal pest that comprises an orally ingestible contraceptive, a pliable bulk feed component, and a casing that encases the soft bait composition.

23. The orally ingestible soft bait of embodiment 22 that comprises an orally ingestible contraceptive.

24. The orally ingestible soft bait of embodiment 22 or 23 that comprises gossypol.

25. The orally ingestible soft bait of embodiment 22, 23 or 24 that comprises 0.0005-0.0045 wt. % free gossypol.

26. The orally ingestible soft bait of any one of embodiments 22-25 that is encased in a casing that protects it from heating, drying, contamination with dirt or soil or by insects or other conditions that would reduce the potency, allure, or edibility of the soft bait compared to an identical soft bait that is not encased.

27. The orally ingestible soft bait of any one of embodiments 22-26, wherein the casing comprises collagen.

28. The orally ingestible soft bait of any one of embodiments 22-27, wherein the casing comprises cellulose, a polysaccharide or cotton or other woven or nonwoven fibrous casing.

29. The orally ingestible soft bait of any one of embodiments 22-28, wherein the casing is a natural casing.

30. The orally ingestible soft bait of any one of embodiments 22-29, wherein the casing is an artificial or synthetic casing.

31. The orally ingestible soft bait of embodiment 22-30, wherein the casing is edible or digestible.

32. The orally ingestible soft bait of embodiment 22-31, wherein the casing is not edible or digestible.

33. The composition of embodiment 22-32, wherein the soft bait is pliable at a temperature of 0, 10, 20, 30, 40, 50 or >50° C.

34. The orally ingestible soft bait of any one of embodiments 22-33, wherein the casing is configured to release alluring odors from the soft bait or comprises a scent or attractant for a rodent or other target animal.

35. The orally ingestible soft bait of any one of embodiments 22-34 that ranges in length from 0.5 to 10 inches and in diameter or average diameter from 15 to 45 mm.

36. The orally ingestible soft bait of any one of embodiments 22-35, wherein the casing membrane ranges from 0.1 to 2.0 mm in thickness or average thickness.

37. The orally ingestible soft bait of any one of embodiments 22-36, wherein the casing membrane ranges from 0.1 to 1.0 mm in thickness or average thickness.

38. A method for reducing a population of rodents or other animal pests comprising placing an orally ingestible soft bait of any one of embodiments 22-37 in a location frequented or accessible to the rodent or other animal pest, wherein, optionally, the soft bait emits an odor that lures or encourages its consumption of the soft bait and administering said soft bait to the rodent or other animal pest upon consumption of the soft bait.

39. A method for making an orally ingestible soft bait composition for a rodent or other animal pest that comprises an orally ingestible contraceptive, a pliable bulk feed component, and a casing that encases the soft bait composition comprising: preparing a soft bait by admixing the orally ingestible contraceptive with a pliable bulk feed ingredient, filling the casing with the soft bait, and tying, fusing, gluing, or otherwise securing the ends of the casing so as to secure the soft bait inside the casing.

40. The method of embodiment 39, wherein the soft bait is encased in the casing by a single stage vacuum stuffer.

41. The method of embodiment 39 or 40, wherein the soft bait is encased in the casing by a dual or multiple stage vacuum stuffer.

42. The method of embodiment 39, 40, or 41, wherein the soft bait is encased in the casing by a horn stuffer, horizontal stuffer, vertical stuffer or hydraulic stuffer.

43. The method of any one of embodiments 39-42 that comprises providing the casing,
providing a vacuum stuffer comprising a filling chamber, a vacuum system, and a stuffer nozzle,
positioning the casing on the stuffer nozzle,
introducing the soft bait into the filling chamber,
activating the vacuum system to apply reduced pressure in the filling chamber,
expelling air from the casing by applying the reduce pressure to the filling chamber, thereby expanding and conforming the casing to the stuffer nozzle,
introducing the soft bait into the casing by applying positive pressure to the filling chamber, thereby forcing the soft bait into the casing,
optionally filling a segment of the casing filed with the first soft bait with a second or subsequent soft bait,
sealing the casing when filled with the soft bait(s),
removing the casing from the stuffer nozzle.

44. The method of any one of embodiments 39-43, wherein the casing comprises collagen.

### Example 1

### Rodent Consumption of Bait Having Free Gossypol Content ≤0.025 wt %

The rodent bait products disclosed herein are designed to specifically appeal to rats with lower gossypol concentrations and desirable ingredients in the form of a soft bait.

Product palatability testing at two field sites revealed high acceptance of low gossypol levels when provided to numerous species of wild rats, including Rattus norvegicus, Rattus rattus, Sigmodon hispidus, and Oryzomys palustris. Baits with higher concentrations of gossypol may still be consumed but will likely make the rats or other rodents sick.

At each site, rats consumed a total of 265 g (18 g/day) and 200 g (13 g/day), respectively, over a 15-day period (see figure below). Rats willingly fed on the bait each day consuming 65% and 90% of available product with no indication of stopping. See FIG. 2.

Gossypol is known to have a cumulative effect and can cause gastrotoxicity at high doses over long exposure periods (Kitada, et al. 2008; Sharma, et al, 1999; Gadelha et al. 2014). Lower doses and shorter exposure durations can prevent unwanted toxic effects while still affecting fertility (Saksena & Salmonsen, 1982).

When administered at concentrations that are relatively high in comparison to the body mass of an animal, gossypol intake could exceed the rate of removal, causing build-up in the body which can reach toxic levels. Differences in the absorption, distribution, biotransformation, and elimination of gossypol also vary largely between species (EFSA).

At high doses in rats, gossypol can result in reduced food intake, anorexia, and hepatic toxicity (Romualdo et al. 2002; Gadelha et al. 2014). From a pest management perspective, it is imperative that our bait is consumed freely by rats at adequate dosing without causing changes in feeding patterns, food preferences, or body mass after acute or chronic exposure to gossypol. Additionally, recovery in fertility has been shown in rats after withdrawal of gossypol treatment (Chang et al. 1982). To maintain results, a lower amount of free gossypol is necessary to prevent side-effects and allow for continuous feeding at lower dosing that is proven to be effective at reducing fertility in rats.

### Example 2

Efficacy Study on Rattus norvegicus Using Soft Bait Containing Cottonseed <0.0025 wt. %

The objective of this study was to determine if rats would voluntarily consume the product and if treatment would impact average litter size and/or the number of births following breeding to show reduced fertility.

Sexually mature Sprague Dawley rats (Rattus norvegicus) were provided 40 g of the soft bait containing cottonseed as active ingredient or control bait (inert only) for 56 days, then paired for breeding based on low or high consumption levels of the active to determine reproductive effects. There was a total of 80 animals (40 males, 40 females) in the study. Control bait containing all of the same inert ingredients but without the active ingredient was used for comparison. Water and chow were provided ad liberum during study and rats were observed and weighed daily to assess their general health.

Following breeding, rats were euthanized and their organs, including reproductive tissue, were necropsied and morphological changes were assessed for signs of toxicity.

Throughout study, male and female rats consumed Evolve each day over the 8 weeks of exposure and did so while chow and water were freely provided, demonstrating palatability and willingness to continuously feed. Males ate an average of 3.42 g of active bait each day or approximately 0.9% of their body weight. Females ate an average of 6.32 g of active each day or 2% of their body weight. All animals were healthy throughout the exposure period and no signs of illness, distress, or behavioral changes were observed. Both control and treated rats consumed consistent amounts of food and water while bait was provided, indicating no side-effects. No animals lost weight during or after exposure to bait.

Rats that consumed active bait showed significantly lower birth rates (p=0.0242) and average number of pups born per litter (p=0.0383) compared to controls. Rats that consumed low and high amounts of active bait saw similar effects on fertility, although those with high consumption had greater significance.

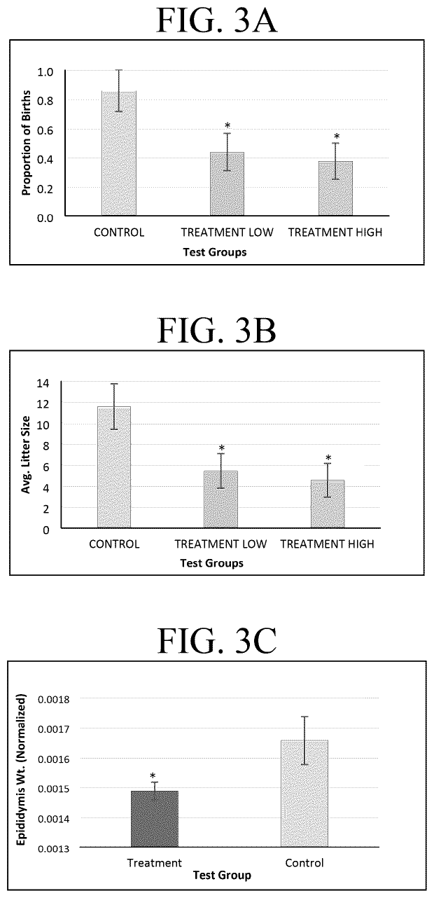

Control females had a birth rate of 86%, while treated females had birth rates of 44% and 38% for the low and high cottonseed consumption groups, respectively. See FIG. 3A.

Female controls had an average of 11.57 pups born per litter, whereas treatment groups with low bait consumption had an average of 5.44 pups and those with high consumption an average of 4.56 pups. See FIG. 3B.

Overall, rats with higher consumption of Evolve bait had 61% reduction in average litter size and rats with lower consumption had 53% reduction compared to controls.

There were no differences in weight of non-reproductive tissues (liver, kidneys, adrenals, spleen, and heart) between rats provided bait or control for either sex. Additionally, there were no morphological changes between test groups, indicating no toxicological effects.

Male reproductive tissue weights for testes and epididymis were examined for comparison between treated and control groups. There were no significant changes in testes, but epididymis weights were significantly reduced in treated males that consumed bait compared to controls. Lower epididymis weights suggest reduced fertility by decrease in sperm density through damage of germ cells. See FIG. 3C.

### Example 3

### Method for Producing a Collagen Casing Containing a Gossypol Soft Bait.

A commercial dehydrated collagen casing is soaked in cold water according to the manufacturer's instructions for about 30 minutes or until pliable.

A soft bait mixture comprising oat flour, cottonseed meal, cottonseed oil, shortening, gossypol at a 0.0045 wt. %, sodium saccharin and citric acid is uniformly mixed at room temperature.

A single stage vacuum stuffer comprising a filling chamber, a vacuum system, and a stuffer nozzle is provided. The hydrated collagen casing is positioned on a stuffer nozzle of the vacuum stuffer and the soft, uniformly mixed soft bait mixture is introduced into the filling chamber and the vacuum system is activated to apply reduced pressure in the filling chamber for a time sufficient for expanding and conforming the casing to the stuffer nozzle. The soft bait is introduced into the collagen casing by applying positive pressure to the filling chamber. Once filled with the soft bait the casing is sealed by twisting closed the open ends of the casing and the stuffed casing is removed from the stuffer nozzle. The filled casing is preferably recovered in a form of one or more links such as those shown in FIGS. 4A-4C.

Drafting Terminology. Terminology used herein is for the purpose of describing particular embodiments only and is not intended to be limiting of the invention.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

As used herein in the specification, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Ranges include endpoints and all subranges and intermediate values.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

The description and any specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears.

The citation of references herein does not constitute an admission that those references are prior art or have any relevance to the patentability of the technology disclosed herein. Any discussion of the content of references cited is intended merely to provide a general summary of assertions made by the authors of the references and does not constitute an admission as to the accuracy of the content of such references.

### REFERENCES

1. Nowzari, F., Rahmanifar, F., Tanideh, N., & Dorvash, M. R. (2021). Dietary cottonseed consumption induced subfertility in male and female rats; a potent eco-friendly rodenticide compound. Research Square 20. <https://doi.org/10.21203/rs.3.rs-538459/v1>
2. Romualdo, G. S., Klinefelter, G. R., & de, K. (2002). Postweaning exposure to gossypol results in epididymis-specific effects throughout puberty and adulthood in rats. Journal of Andrology, 23 (2), 220-228.
3. Gadelha, I. C., de Macedo, M. F., Oloris, S. C., Melo, M. M., & Soto-Blanco, B. (2014). Gossypol promotes degeneration of ovarian follicles in rats. Scientific World Journal, 986184. <https://doi.org/10.1155/2014/986184>
4. Lin, Y. C., Fukaya, T., Rikihisa, Y., & Walton, A. (1985). Gossypol in female fertility control: ovum implantation and early pregnancy inhibited in rats. Life Sciences, 37 (1), 39-47. <https://doi.org/10.1016/0024-3205 (85) 90623-x>
5. Luz, V. B., Gadelha, I. C. N., Cordeiro, L. A. V., Melo, M. M., & Soto-Blanco, B. (2018). In vitro study of gossypol's ovarian toxicity to rodents and goats. Toxicon, 145, 56-60.
6. Ding, Z. M., Chen, Y. W., Wang, Y. S., Ahmad, M. J., Yang, S. J., Duan, Z. Q., Liu, M., Yang, C. X., Xiong, J. J., Liang, A. X., & Huo, L. J. (2021). Gossypol exposure induces mitochondrial dysfunction and oxidative stress during mouse oocyte in vitro maturation. Chemico-Biological Interaction, 348, 109642.
7. Sein G. M. (1986). The embryotoxic and immunodepressive effects of gossypol. The American Journal of Chinese Medicine, 14 (3-4), 110-115.
8. Li, F., Booth, G. M., & Seegmiller, R. E. (1989). Evidence for embryotoxicity of gossypol in mice and chicks with no evidence of mutagenic activity in the ames test. Reproductive Toxicology, 3 (1), 59-62.
9. Lin, Y. C., Rajamahendran, R., Rikihisa, Y., & Gwazdauskas, F. C. (1989). Effect of gossypol and its metabolite on in vitro early development of mouse embryos. Theriogenology, 31 (4), 727-740.
10. Randel, R. D., Chase, C. C. Jr., & Wyse, S. J. (1992). Effects of gossypol and cottonseed products on reproduction of mammals. Journal of Animal Science, 70 (5), 1628-1638.
11. El-Sharaky, A. S., Newairy, A. A., Elguindy, N. M., & Elwafa, A. A. (2010). Spermatotoxicity, biochemical changes and histological alteration induced by gossypol in testicular and hepatic tissues of male rats. Food and Chemical Toxicology, 48 (12), 3354-3361.
12. Chris-ozoko, L. E., Okpe, O., Iju, W. J., & Oyem, J. C. (2018). Histomorphological effects of cottonseed oil on testes in adult male Wistar rats. Galician Medical Journal, 25 (4).
13. Fornés, M. W., Barbieri, A. M., & Burgos, M. H. (1993). Sperm motility loss induced by gossypol: relation with OH· scavengers, motile stimulators and malondialdehyde production. Biochemical and Biophysical Research Communications, 195 (3), 1289-1293. <https://doi.org/10.1006/bbrc.1993.2183>
14. Ali, I. N., Al-Obaidi, S. A., & Fakhrildin, M. B. M. (2010). Effect of aqueous and alcoholic extracts of cottonseed on some parameters of sperm and reproductive performance of albino mice. Al-Nahrain Journal of Science, 13 (4), 151-158.
15. Singla, N. & Garg, M. (2013). Effect of crude cottonseed oil containing gossypol on fertility of male and estrous cycle of female Bandicota bengalensis Gray and Hardwicke. Journal of Applied Animal Research, 41 (2), 156-165.
16. Singh, S. K., & Rath, S. K. (1990). Histologic changes in the mouse testis after treatment with gossypol tetra-acetic acid. Archives of Histology and Cytology, 53 (4), 393-396.
17. Hunt, S., & Mittwoch, U. (1984). Effects of gossypol on sperm counts in two inbred strains of mice. Journal of reproduction and fertility, 70 (1), 341-345.
18. Nadakavukaren, M. J., Sorensen, H. R., & Tone, J. N. (1979). Effect of gossypol on the ultrastructure of rat spermatozoa. Cell and Tissue Research, 204 (2), 293-296.
19. Farrag A. K., Ismail, H. I., Khalifa, E. A., & El-Gamal, M. M. The possible synergistic effect of gossypol (cottonseed oil) and Schistosomiasis mansoni on the male reproductive organs in experimenting infected mice. (2007). Tanta Medical Sciences Journal, 2 (1), 78-91.
20. Chang, C. C., Gu, Z. P., & Tsong, Y. Y. (1982). Studies on gossypol toxicity, antifertility, and endocrine analyses in male rats. International Journal of Fertility 27 (4), 213-218.
21. Coulson, P. B., Snell, R. L., & Parise, C. (1980). Short term metabolic effects of the antifertility agent, gossypol, on various reproductive organs of male mice. International Journal of Andrology, 3 (5), 507-518.

1. A soft bait composition comprising an amount of free gossypol sufficient to induce contraception or decrease fertility in a rodent when orally ingested; and an edible carrier or bulk feed component.

2. The soft bait composition of claim 1, wherein the soft bait composition is pliable at a temperature of 0, 10, 20, 30, 40, 50 or >50° C. and has a Shore 00 value of no more than 50.

3. The soft bait composition of claim 1 wherein a content of free gossypol ranges from 0.0005-0.0045 wt. %.

4. The soft bait composition of claim 1, wherein the free gossypol concentration ranges from 0.0005 to 0.001 wt. %.

5. The soft bait composition of claim 1, wherein the free gossypol content is derived from cottonseed oil.

6. The soft bait composition of claim 1 that is encased in a casing that protects it from heating, drying, contamination with dirt or soil or from insects or other conditions that would reduce the potency, allure, or edibility of the soft bait compared to an identical soft bait that is not encased.

7. The orally ingestible soft bait of claim 6, wherein the casing comprises collagen, cellulose, or a woven or non-woven fabric.

8. The orally ingestible soft bait of claim 6, wherein the casing is a natural casing.

9. The composition of claim 1, comprising:
0.0005 to 0.0045 wt. % free gossypol,
60-80 wt. % oat flour,
1 to 10 wt. % cottonseed meal,
0.05 to 1% cottonseed oil,
20 to 30 wt. % shortening,
0.1 to 5.0 wt. % sodium saccharin or other non-nutritive sweetener, and
0.05 to 0.25 wt. % citric acid or other preservative.

10. The composition of claim 1, comprising:
0.0005 to 0.0045 wt. % free gossypol,
60-80 wt. % oat flour,
1 to 10 wt. % cottonseed meal,
0.05 to 1% cottonseed oil,
20 to 30 wt. % shortening,
0.1 to 5.0 wt. % sodium saccharin, and
0.5 to 0.25 wt. % citric acid.

11. A method for inducing contraception or for reducing fertility in a rodent comprising administering the soft bait composition claim 1 to a targeted rodent.

12. The method of claim 11, wherein said administering comprises placing the composition at a location accessible to its consumption by the targeted rodent.

13. The method of claim 11, wherein the targeted rodent is Rattus norvegicus, Rattus rattus, Sigmodon hispidus, or Oryzomys palustris.

14. The method of claim 11, wherein the targeted rodent is Mus musculus, Peromyscus maniculatus, Peromyscus leucopus, Reithrodontomys megalotis, or Peromyscus gossypinus.

15. The method of claim 11, wherein an amount of free gossypol sufficient to reduce sperm count, sperm motility, or sperm morphology score in the male rodent consuming or repeatedly consuming the composition.

16. The method of claim 11, wherein an amount of free gossypol sufficient to disrupt estrous cycle, reduce ovarian follicles, or impair early embryonic development and pregnancy in the female rodent consuming or repeatedly consuming the composition.

17. The method of claim 11, wherein the soft bait composition is administered in the form of a paste or block.

18. The method of claim 11, wherein the soft bait composition is administered in an encased form, wherein the composition is surrounded by an encasement.

19. A method for reducing a population of rodents or other animal pests comprising placing an orally ingestible soft bait of claim 1 in a location frequented or accessible to the rodent or other animal pest, wherein the soft bait emits an odor that lures or encourages its consumption of the soft bait thereby administering said soft bait to the rodent upon consumption of the soft bait.

20. A method for making an orally ingestible soft bait composition for a rodent or other animal pest that comprises an orally ingestible contraceptive, a pliable bulk feed component, and a casing that encases the soft bait composition comprising:
preparing a soft bait by admixing the orally ingestible contraceptive with a pliable bulk feed ingredient, and
filling the casing with the soft bait, and tying, fusing, gluing, or otherwise securing the ends of the casing so as to secure the soft bait inside the casing;
thereby making the orally ingestible soft bait composition.
